(19) United States
(12) Patent Application Publication
(10) Pub. No.: US 2004/0039021 A1
(43) Pub. Date: Feb. 26, 2004

(54) Pharmaceutical anti-inflammatory aerosol formulation

(76) Inventors: Duncan Robert Armour, Hertfordshire (GB); David Brown, Herfodshire (GB); Miles Congreve, Cambridgeshire (GB); Paul Gore, Hertfordshire (GB); Darren Victor, Steven Green, Hertfordshire (GB); Stuart Holman, Hertfordshire (GB); Torquil Iain, Maclean Jack, Hertfordshire (GB); Steven Philip Keeling, Hertfordshire (GB); Andrew McMurtrie Mason, Hertfordshire (GB); Karen Morriss, Hertfordshire (GB); Nigel Grahame Ramsden, Hertfordshire (GB); Marian Thomas, Hertfordshire (GB); Peter Ward, Hertfordshire (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/311,556

(22) PCT Filed: Jun. 15, 2001
(86) PCT No.: PCT/GB01/02634

(30) Foreign Application Priority Data
Jun. 16, 2000 (GB) 0014849.4

(51) Int. Cl. A61K 31/445; A61L 9/04

(52) U.S. Cl. 514/317; 424/45

(57) ABSTRACT

The present invention relates to a pharmaceutical aerosol formulation comprising a hydrofluoroalkane (HFA) propellant having suspended therein particulate (2S)-3-[4-({[4-(aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof. Methods and uses of the formulation in the treatment of respiratory disorders are also described, as are canisters and metered dose inhalers containing said formulation.

The present invention relates to a pharmaceutical formulation for use in the administration of medicaments by inhalation. In particular, this invention relates to a pharmaceutical formulation for use in pressurised metered dose inhalers (MDI's). The invention also relates to methods for their preparation and to their use in therapy.

Inhalers are well known devices for administering pharmaceutically active materials to the respiratory tract by inhalation. Such active materials commonly delivered by inhalation include bronchodilators such as β32 agonists and anticholinergics, corticosteroids, anti-allergics and other materials that may be efficiently administered by inhalation, thus increasing the therapeutic index and reducing side effects of the active material.

(2S)-3-[4-({[4-(Aminocarbonyl)-1 -piperidinyl]carbonyl}oxy)phenyl]-2-[((2S)-4-methyl-2-{[2-(2-methylphenoxy)acetyl]amino}pentanoyl)amino] propanoic acid has recently been disclosed in International Patent Application (PCT/EP99/10000) as a novel antagonist of both α4β1 and α4β7 integrins which, as a consequence, results in effective anti-inflammatory properties.

Metered dose inhalers (MDIs) are the most common type of a wide range of inhaler types and utilise a liquefied propellant to expel droplets containing the pharmaceutical product to the respiratory tract as an aerosol. MDI formulations are generally characterised as solution formulations or suspension formulations.

The most commonly used aerosol propellants for medicaments have been Freon 11 (CCl₃F) in admixture with Freon 12 (CCl₂F₂) and Freon 114 (CF₂Cl.CF₂Cl). However, these propellants are now believed to provoke the degradation of stratospheric ozone and their use is now being phased out to eliminate the use of all CFC containing aerosol propellants. There is thus a need to provide an aerosol formulation for medicaments which employ so called ‘ozone-friendly’ propellants.

Hydrofluoroalkanes (HFAs; known also as hydrofluorocarbons or HFCs) contain no chlorine and are considered less destructive to ozone and these are proposed substitutes for CFCs. In particular, 1,1,1,2-tetrafluoroethane (HFA 134a) and 1,1,1,2,3,3,3-heptafluoropropane (HFA 227) have been acknowledged to be the best candidates for non-CFC propellants.

The efficiency of an aerosol device, such as an MDI, is a function of the dose deposited at the appropriate site in the lungs. Deposition is affected by several factors, of which one of the most important is the aerodynamic particle size. Solid particles and/or droplets in an aerosol formulation can be characterised by their mass median aerodynamic diameter (MMAD, the diameter around which the mass aerodynamic diameters are distributed equally).

Particle deposition in the lung depends largely upon three physical mechanisms:

1. impaction, a function of particle inertia;

2. sedimentation due to gravity; and

3. diffusion resulting from Brownian motion of fine, submicrometer (<1 μm) particles.

The mass of the particles determines which of the three main mechanisms predominates.

The effective aerodynamic diameter is a function of the size, shape and density of the particles and will affect the magnitude of forces acting on them. For example, while inertial and gravitational effects increase with increasing particle size and particle density, the displacements produced by diffusion decrease. In practice, diffusion plays little part in deposition from pharmaceutical aerosols. Impaction and sedimentation can be assessed from a measurement of the

MMAD which determines the displacement across streamlines under the influence of inertia and gravity, respectively.

Aerosol particles of equivalent MMAD and GSD (geometric standard deviation) have similar deposition in the lung irrespective of their composition. The GSD is a measure of the variability of the aerodynamic particle diameters.

For inhalation therapy there is a preference for aerosols in which the particles for inhalation have a diameter of about 0.5 to 5 μm. Particles which are larger than 5 μm in diameter are primarily deposited by inertial impaction in the orthopharynx, particles 0.5 to 5 μm in diameter, influenced mainly by gravity, are ideal for deposition in the conducting airways, and particles 0.5 to 31 μm in diameter are desirable for aerosol delivery to the lung periphery. Particles smaller than 0.5 μm may be exhaled.

Thus, according to the present invention we provide a pharmaceutical aerosol formulation, comprising a hydrofluoroalkane (HFA) propellant having suspended therein particulate (2S)-3-[4-({[4-(Aminocarbonyl)-1-piperidinyl]carbonyl}oxy)phenyl]-2-[((2S)-4-methyl-2-{[2-(2 methylphenoxy)acetyl]amino}pentanoyl)amino] propanoic acid or a salt or solvate thereof.

Examples of suitable salts include physiologically acceptable salts such as alkali metal salts, for example calcium, sodium and potassium salts and salts with (trishydroxymethyl)aminomethane.

Preferably, the (2S)-3-[4-({[4-(Aminocarbonyl)-1-piperidinyl]carbonyl}oxy)phenyl]2-[((2S)-4-methyl-2-{[2-(2-methylphenoxy)acetyl]amino}pentanoyl)amino] propanoic acid will be present as the free acid. The potassium salt is also of interest.

Optionally a further particulate active ingredient suitable for inhalation therapy may be incorporated into the formulation such as a corticosteroid (eg fluticasone propionate) or a bronchodilator (eg salmeterol or albuterol or a salt thereof).

We prefer that the mass median diameter (MMD) of the (2S)-3-[4-({[4(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof is between 1 and 10 μm, most preferably between 2 and 5 μm.

To achieve these particle sizes the particles of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof as produced may be size reduced by conventional means eg. by micronisation. The desired fraction may be separated out by air classification or sieving. Preferably, the particles will be crystalline, prepared for example by a process which comprises mixing in a continuous flow cell in the presence of ultrasonic radiation a flowing solution of (2S)-3-[4-({[4-(Aminocarbonyl)-1-piperidinyl]carbonyl}oxy)phenyl]-2-[((2S)4-methyl-2-{[2-(2-methylphenoxy)acetyl]amino}pentanoyl) amino] propanoic acid (or a salt or solvate thereof as medicament in a liquid solvent with a flowing liquid antisolvent for said medicament (as described in International Patent Application PCT/GB99/04368).

Examples of HFA propellants include 1,1,1,2-tetrafluoroethane (HFA134a) and 1,1,1,2,3,3,3-heptafluoro-n-propane (HFA227) and mixtures thereof. The preferred propellant is 1,1,1,2-tetrafluoroethane (HFA134a). 1,1,1,2,3,3,3-heptafluoro-n-propane (HFA227) is also of particular interest.

Formulations may optionally contain a surfactant. The surfactant must be physiologically acceptable when it is used by inhalation. Examples of surfactants which can be used according to the present invention are conventional surfactants including anionic surfactants such as oleic acid, non-ionic surfactants such as sorbitan trioleate, sorbitan monooleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of ethylene oxide and of propylene oxide, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400 or glyceryl monolaurate, or cationic surfactants, such as cetylpyridinium chloride or benzalkonium chloride. Other examples of surfactants include synthetic phosphatides eg. distearoylphosphatidylcholine. When conventional surfactants are employed it will generally be necessary to incorporate a compound of higher polarity than the propellant (eg ethanol) in order to assist solubilisation of the surfactant in the propellant.

Preferred conventional surfactants include lecithin, oleic acid and sorbitan trioleate.

Alternative surfactants include fluorinated surfactants such as those described in WO96/09816, polyethoxylated surfactants such as those described in WO92/00061, polymers such as PVP and methacrylates such as those described in WO93/05765, surfactants comprising a chain of units derived from a hydroxy acid, amino acid or mercapto acid (eg oligolactic acid) such as those described in WO94/21229 and surfactants comprising a chain of diol/diacid condensate units such as those described in WO94/21228. Oligolactic acid is of particular interest. The above mentioned alternative propellants may desirably employed without the need for use of any higher polarity additive, although the use of such is nevertheless not excluded.

Preferably, the surfactant will be present within the formulation at an amount between 0.01 and 20% (w/w), most preferably 0.1 to 5% (w/w), especially 0.5 to 2% (w/w).

A higher-polarity additive may be employed if needed in a concentration of, say, up to 10% eg 0.1-10%, especially 0.1-5% however the concentration should not be so high that solubilisation of the active ingredient in the formulation gives rise to Ostwald ripening and particle size growth.

Preferably the use of a higher-polarity additive is avoided. Preferably also use of a surfactant is avoided. We prefer a pharmaceutical aerosol formulation which consists essentially of (especially a formulation which consists of) a hydrofluoroalkane (HFA) propellant having suspended therein particulate (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof. In the case of a further active ingredient being present we prefer a pharmaceutical aerosol formulation which consists essentially of (especially a formulation which consists of) a hydrofluoroalkane (HFA) propellant having suspended therein particulate (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof and a further particulate active ingredient suitable for inhalation therapy.

The formulation according to the invention will be used in association with a suitable metering valve. We prefer that the formulation is actuated by a metering valve capable of delivering a volume of between 50 μl and 100 μl, eg 50 μl or 63 μl or 100 μl.

The pharmaceutical composition according to the present invention may be filled into canisters suitable for delivering pharmaceutical aerosol formulations.

Canisters generally comprise a container capable of withstanding the vapour pressure of the HFA propellant, such as plastic or plastics coated glass bottle or preferably a metal can, for example an aluminium can which may optionally be anodised, lacquer-coated and/or plastics coated, which container is closed with a metering valve. It may be preferred that canisters be coated with a fluorocarbon polymer as described in WO 96/32151, for example, a co-polymer of polyethersulphone (PES) and polytetrafluoroethylene (PTFE). Another polymer for coating that may be contemplated is FEP (fluorinated ethylene propylene). The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in WO92/11190 and valves containing EPDM rubber as described in WO95/02651 are especially suitable. Suitable valves are commercially available from manufacturers well known in the aerosol industry, for example, from Valois, France (eg. DF10, DF30, DF60), Bespak plc, UK (eg. BK300, BK356, BK357) and 3M-Neotechnic Ltd, UK (eg. Spraymiser™). The DF31 valve of Valois, France is also suitable.

Valve seals, especially the gasket seal, will preferably be manufactured of a material which is inert to and resists extraction into the contents of the formulation, especially when the contents include ethanol.

Valve materials, especially the material of manufacture of the metering chamber, will preferably be manufactured of a material which is inert to and resists distortion by contents of the formulation, especially when the contents include ethanol. Particularly suitable materials for use in manufacture of the metering chamber include polyesters eg polybutyleneterephthalate (PBT) and acetals, especially PBT.

Materials of manufacture of the metering chamber and/or the valve stem may desirably be fluorinated, partially fluorinated or impregnated with fluorine containing substances in order to resist drug deposition.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The medicament is added to a charge vessel and a mixture of ethanol, low volatility component and liquefied propellant is pressure filled through the charge vessel into a manufacturing vessel. An aliquot of the formulation is then filled through the metering valve into the canister. Typically, in batches prepared for pharmaceutical use, each filled canister is check-weighed, coded with a batch number and packed into a tray for storage before release testing.

In an alternative process, an aliquot of the liquified formulation is added to an open canister under conditions which are sufficiently cold that the formulation does not vaporise, and then a metering valve crimped onto the canister.

Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise, for example a valve actuator and a cylindrical or cone-like passage through which medicament may be delivered from the filled canister via the metering valve to the nose or mouth of a patient eg. a mouthpiece actuator. Metered dose inhalers are designed to deliver a fixed unit dosage of medicament per actuation or ‘puff’, for example in the range of 10 to 5000 μg medicament per puff.

In a typical arrangement the valve stem is seated in a nozzle block which has an orifice leading to an expansion chamber. The expansion chamber has an exit orifice which extends into the mouthpiece. Actuator (exit) orifice diameters in the range 0.2-0.45 mm are generally suitable eg 0.22, 0.25, 0.30, 0.33 or 0.42 mm.

Actuator jet lengths are typically in the range 0.30-1.7 mm eg 0.30, 0.65 or 1.50 mm.

Preferably, the dose of (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or salt or solvate thereof will be between 0.1 and 10 mg per day, most preferably between 0.5 and 3 mg.

Metered dose inhalers are designed to deliver a fixed unit dosage of medicament per actuation or ‘puff’, for example in the range of 25 to 300 μg medicament per actuation. We prefer the formulation to be suitable for delivering a therapeutic amount of drug in one or two actuations.

The concentration of drug in the formulation will therefore typically be in the range 0.02 to 5% w/w.

Typically, administration may be one or more inhalations (eg. 1, 2, 3 or 4 inhalations) up to five times per day.

Administration of medicament may be indicated for the treatment of mild, moderate or severe acute or chronic symptoms or for prophylactic treatment. It will be appreciated that the precise dose administered will depend upon the age and condition of the patient, the quantity and frequency of administration will ultimately be at the discretion of the attendant physician.

The filled canisters and metered dose inhalers described herein comprise further aspects of the present invention.

A still further aspect of the present invention comprises a method of treating respiratory disorders which comprises administration by inhalation of an effective amount of a formulation herein before described.

Preferably, the respiratory disorder will be asthma. Allergic rhinitis is also of interest. It will be appreciated that when the respiratory disorder is allergic rhinitis the formulation of the present invention will be delivered via the nasal route.

A further aspect of the present invention comprises the use of a formulation herein before described in the manufacture of a medicament for the treatment of respiratory disorders, eg. asthma or allergic rhinitis.

The invention may be illustrated by the following non-limiting examples:

### EXAMPLE A

### (2S)-3-[4-({[4-(Aminocarbonyl)-1-piperidinyl]carbonyl} oxy) phenyl]-2-[((2S)-4-methyl-2-{[2-(2-methylphenoxy)acetyl]amino}pentanoyl) amino] Propanoic Acid

To Wang resin (50 g) was added a solution of (2S)-3-[4-(allyloxy)phenyl]-2-[(tert-butoxycarbonyl)amino]propanoic acid (115.8 g) and 1-hydroxybenzotriazole (48.6 g) in DMF (475 ml). After 15 minutes 1,3-diisopropylcarbodiimide (56.5 ml) was added and the mixture was stirred for 24 h at 45° C. The resin was filtered and washed with DMF (3×360 ml), methanol (3×360 ml) and dichloromethane (3×700 ml). To a slurry of the resin in dichloromethane (644 ml) was added pyridine (14.7 ml). Acetic anhydride (26.9 ml) was added and the mixture was stirred for 12 h at 20° C. The resin was filtered and washed with dichloromethane (3×550 ml), methanol (3×370 ml) and dichloromethane (3×550 ml). A slurry of 20 g of the resin in dichloromethane (100 ml) was cooled to 2-5° C. and treated with a solution of phenol (20 g) in dichloromethane (80 ml). Chlorotrimethylsilane (20 ml) was added dropwise and the mixture was stirred for 6 h at 2-5° C. The resin was filtered and washed with dichloromethane (3×200 ml), methanol (3×200 ml), 10% water in DMF (2×200 ml), 10% diisopropylethylamine in DMF (3×200 ml), DMF (200 ml), methanol (3×200 ml) and dichloromethane (3×200 ml).

A slurry of the resin in DMF (55 ml) was treated with a solution of Fmoc-leucine (32.7 g) and 1-hydroxybenzotriazole (12.5 g) in DMF (85 ml). After 5 minutes 1,3-diisopropylcarbodiimide (19.3 ml) was added and the mixture was stirred for 15 h at 20° C. The resin was filtered and washed with DMF (3×150 ml), methanol (3×150 ml) and dichloromethane (3×150 ml).

The resin was treated with 20% piperidine in DMF (180 ml) and stirred for 1 h at 20° C. The resin was filtered and washed with DMF (3×150 ml), dichloromethane (3×150 ml), DMF (3×150 ml) and dichloromethane (3×150 ml). To a slurry of this in DMF (50 ml) was added a solution of (2-methylphenoxy)acetic acid (17.9 g) and 1-hydroxybenzotriazole (14.6 g) in DMF (100 ml). After 5 minutes 1,3-diisopropylcarbodiimide (16.9 ml) was added and the mixture was stirred for 65 h at 20° C. The resin was filtered and washed with DMF (2×150 ml), methanol (3×150 ml) and dichloromethane (3×150 ml).

A slurry of the resin in dichloromethane (60 ml) was treated with a solution of tetrakis(triphenylphosphine)palladium(0) (5.21 g) in dichloromethane (140 ml) followed by morpholine (13 ml). The mixture was stirred for 2 h at 20° C. then the resin was filtered and washed with dichloromethane (7×200 ml).

A slurry of the resin in dichloromethane (160 ml) was treated with diisopropylethylamine (12.4 ml) followed by 4-nitrophenyl chloroformate (24.8 g) in 3 portions at 5 minute intervals. The mixture was stirred for 1 h at 20° C. The resin was filtered and washed with dichloromethane (3×200 ml). The resin was treated with a solution of isonipecotamide (15.8 g) in DMF (180 ml) and the mixture was stirred for 1.5 h at 20° C. The resin was filtered and washed with DMF (4×200 ml) and dichloromethane (2×200 ml).

The resin was treated with 50% TFA in dichloro methane (200 ml). After stirring for 1 h at 20° C. the resin was filtered and washed with dichloromethane (5×200 ml). The combined filtrate and washings were evaporated in vacuo. The residue was azeotroped with toluene (2×100 ml) then triturated with ether (50 ml) and the resulting white solid filtered. To this was added acetonitrile (150 ml) and the mixture was heated to, reflux. The resulting suspension was allowed to cool to 20° C. and stirred for 18 h. The mixture was filtered to give the title compound as a white solid (4.9 g).

### EXAMPLE B

### (2S)-3-[4-({[4-(Aminocarbonyl)-1-piperidinyl]carbonyl} oxy) phenyl]-2-[((2S)-4-methyl-2-{[2-(2-methylphenoxy)acetyl]amino}pentanoyl) amino] Propanoic Acid Potassium Salt

A suspension of (2S)-3-[4-({[4-(Aminocarbonyl)-1-piperidinyl]carbonyl} oxy) phenyl]-2-[((2S)-4-methyl-2-{[2-(2-methylphenoxy) acetyllamino}pentanoyl) amino] propanoic acid (10 g) in methanol (150 ml) was warmed to reflux to obtain a clear solution. To this was added a solution of potassium carbonate (1.16 g) in water (7.5 ml). After heating under reflux for two minutes the solvents were evaporated in vacuo to give a crisp foam. To this was added acetonitrile (100 ml) and the mixture was warmed to reflux, during which time the foam collapsed and started to crystallise. After ten minutes the mixture was allowed to cool to 20° C. then filtered under reduced pressure, washed with acetonitrile (25 ml) and ether (50 ml) to give the title compound as a white solid (10.65 g, 100%).

### EXAMPLE 1

An aluminium canister was filled with a formulation as follows:

(2S)-3-[4-({[4-(Aminocarbonyl)-1 -piperidinyl]carbonyl}oxy)phenyl]-2-[((2S)-4-methyl-2-{[2-(2-methylphenoxy)acetyl]amino}pentanoyl)amino] propanoic acid

(prepared according to Example A) 1% w/w

1,1,1,2-tetrafluoroethane: to 100%

in an amount suitable for 120 actuations and the canister was fitted with a metering valve.

### EXAMPLE 2

An aluminium canister was filled with a formulation as follows:

(2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid

potassium salt (prepared according to Example B) 1% w/w

1,1,1,2-tetrafluoroethane: to 100%

in an amount suitable for 120 actuations and the canister was fitted with a metering valve.

### EXAMPLE 3

An aluminium canister was filled with a formulation as follows:

(2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid

(prepared according to Example A) 3% w/w

1,1,1,2-tetrafluoroethane: to 100%

in an amount suitable for 120 actuations and the canister was fitted with a metering valve.

### EXAMPLE 4

An aluminium canister was filled with a formulation as follows:

(2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid

potassium salt (prepared according to Example B) 3% w/w

1,1,1,2-tetrafluoroethane: to 100%

in an amount suitable for 120 actuations and the canister was fitted with a metering valve.

### EXAMPLES 5 AND 6

Aluminium canisters were filled with formulations as follows:

(2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid

(prepared according to Example A): 1 or 3% (w/w)

oligolactic acid: 1% (w/w)

1,1,1,2-tetrafluoroethane: to 100%

in an amount suitable for 120 actuations and the canister was fitted with a metering valve.

### EXAMPLES 7 AND 8

Aluminium canisters were filled with formulations as follows:

(2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid

potassium salt (prepared according to Example B): 1 or 3% (w/w)

oligolactic acid: 1% (w/w)

1,1,1,2-tetrafluoroethane: to 100%

in an amount suitable for 120 actuations and the canister was fitted with a metering valve.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The contents of the above mentioned patent applications are herein incorporated by reference.

1. A pharmaceutical aerosol formulation comprising a hydrofluoroalkane (HFA) propellant having suspended therein particulate (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

2. A pharmaceutical aerosol formulation according to claim 1 wherein the (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid is present as the free acid.

3. A pharmaceutical aerosol formulation according to claim 1 wherein the (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid is present as the potassium salt.

4. A formulation according to claim 1 wherein the hydrofluoroalkane (HFA) propellant is 1,1,1,2-tetrafluoroethane (HFA134a) or 1,1,1,2,3,3,3-heptafluoro-n-propane (HFA227) or a mixture thereof.

5. A formulation according to claim 4 wherein the hydrofluoroalkane (HFA) propellant is 1,1,1,2-tetrafluoroethane (HFA134a).

6. A formulation according to any one of claims 1 to 5 which additionally contains a surfactant.

7. A formulation according to claim 6 wherein the surfactant is selected from: anionic surfactants such as oleic acid, non-ionic surfactants such as sorbitan trioleate, sorbitan monooleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of ethylene oxide and of propylene oxide, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400 or glyceryl monolaurate, or cationic surfactants, such as cetylpyridinium chloride or benzalkonium chloride. Other examples of surfactants include synthetic phosphatides eg. distearoylphosphatidylcholine.

8. A formulation according to claim 7 wherein the surfactant is selected from lecithin, oleic acid and sorbitan trioleate.

9. A formulation according to claim 6 wherein the surfactant is oligolactic acid.

10. A formulation according to any one of claims 6 to 9 wherein the surfactant is present within the formulation at an amount of between 0.01 and 20% (w/w).

11. A formulation according to claim 10 wherein the surfactant is present within the formulation at an amount of between 0.1 and 5% (w/w).

12. A formulation according to claim 11 wherein the surfactant is present within the formulation at an amount of between 0.5 and 2% (w/w).

13. A pharmaceutical aerosol formulation consisting essentially of a hydrofluoroalkane (HFA) propellant having suspended therein particulate (2S)-3-(4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

14. A pharmaceutical aerosol formulation consisting of a hydrofluoroalkane (HFA) propellant having suspended therein particulate (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof.

15. A formulation according to any one of claims 1 to 14 wherein the (2S)-3-[4-({[4-(Aminocarbonyl)-1-piperidinyl]carbonyl}oxy)phenyl]-2-[((2S)-4-methyl-2-{[2-(2-methylphenoxy)acetyl]amino}pentanoyl)amino] propanoic acid or a salt or solvate thereof is present within the formulation in an amount of between 0.02 and 5% (w/w).

16. A canister closed with a metering valve and containing a pharmaceutical aerosol formulation according to any one of claims 1 to 15.

17. A metered dose inhaler which comprises a canister as claimed in claim 16 fitted into a suitable channelling device.

18. A method of treating respiratory disorders which comprises administration by inhalation of an effective amount of a pharmaceutical aerosol formulation according to any one of claims 1 to 15.

19. A method of treating asthma which comprises administration by inhalation of an effective amount of a pharmaceutical aerosol formulation according to any one of claims 1 to 15.

20. A method of treating allergic rhinitis which comprises administration via the nasal route of an effective amount of a pharmaceutical aerosol formulation according to any one of claims 1 to 15.

21. Use of a pharmaceutical aerosol formulation according to any one of claims 1 to 15 in the manufacture of a medicament for the treatment of respiratory disorders, eg. asthma or allergic rhinitis.
